(19) United States
(12) Patent Application Publication
(10) Pub. No.: US 2025/0012186 A1
(43) Pub. Date: Jan. 9, 2025

(54) DETERMINING THRESHOLD HYDRAULIC GRADIENT FOR CAPROCKS ASSOCIATED WITH GEOLOGICAL CO2 AND H2 STORAGE

(71) Applicant: SAUDI ARABIAN OIL COMPANY, Dhahran (SA)

(72) Inventors: Hui-Hai LIU, Houston, TX (US); Jilin Jay ZHANG, Houston, TX (US)

(73) Assignee: SAUDI ARABIAN OIL COMPANY, Dhahran (SA)

(21) Appl. No.: 18/348,427

(22) Filed: Jul. 7, 2023

(51) Int. Cl. E21B 49/02; E21B 25/08

(52) U.S. Cl. CPC E21B 49/02; E21B 25/08

(57) ABSTRACT

Apparatus and methods for determining caprock integrity. The testing apparatus for performing the method including a core container in fluid communication with an upstream reservoir and an upstream pump, and further in fluid communication with a downstream reservoir and a downstream pump. The method including determining threshold hydraulic gradient of a caprock core sample using the testing apparatus.

### FIELD OF INVENTION

The present disclosure relates generally to methods and systems for use in the oil and gas industry, and more particularly, to methods and systems for CO₂ and H₂ sequestration and storage.

### BACKGROUND

Long-term geological sequestration and storage of CO₂, H₂, and other gasses in subterranean formation reservoirs (e.g., saline aquifers or depleted oil and gas reservoirs) offers the possibility of sustaining access to fossil fuels while reducing gaseous emissions that contribute to global warming and climate change. However, prior to implementation, associated risks of gaseous leakage must be carefully assessed to ensure environmental safety. In such reservoirs, where gases are injected for sequestration and storage, caprock integrity is thus a critical consideration, but also a complex geo-mechanical issue. The term “caprock,” and grammatical variants thereof, refers to a layer of hard, generally (preferably) less permeable rock overlying and sealing a deposit of a gas (although other fluids can also be sealed thereunder).

Caprock integrity is particularly significant as stress state changes occur throughout the injection/production life. Evaluation and understanding of mechanical and flow properties of caprock are critical to the design of virtually any project involving fluid injection operations (e.g., CO₂, H₂, and the like). For example, caprock integrity is a driver of success in CO₂ and H₂ sequestration (storage) projects. It is also an integral part in underground storage (e.g., natural gas in salt caverns). In such cases, characterization of geologic, flow, and geo-mechanical properties of the caprock is essential.

Analysis of caprock integrity depends on an integration of disparate data. Conventionally, sparse data (e.g., limited core, mini-fracturing analysis, Brazilian tensile test on core samples, and the like), collected over limited locations and averaged across the caprock thickness was frequently used to quantify the integrity of caprock. However, such approaches, which are typically conducted on rocks prior to production/injection operations, generally result in a high level of uncertainty when determining caprock integrity. Other approaches aimed at decreasing uncertainty often require a suspension of drilling operations to collect data, thereby impacting production and hydrocarbon recovery.

In view of the above, it is desirable that an accurate assessment of caprock integrity be available for site selection, characterization, and operational evaluation.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In one or more aspects, the present disclosure provides a method comprising: collecting a caprock core sample; determining threshold hydraulic gradient for the caprock core sample using a testing apparatus, the testing apparatus comprising: a core container comprising: an upstream inlet in fluid communication with the core container; a downstream inlet in fluid communication with the core container; and a confining pressure pump in fluid communication with a bottom portion of the core container; an upstream reservoir in fluid communication with the upstream inlet of the core container and comprising a first upstream valve for selectively permitting or ceasing fluid flow between the upstream reservoir and the core container; an upstream pump in fluid communication with the upstream reservoir and comprising a second upstream valve for selectively permitting or ceasing fluid flow between the upstream pump and the upstream reservoir; a downstream reservoir in fluid communication with the downstream inlet of the core container and comprising a first downstream valve for selectively permitting or ceasing fluid flow between the downstream reservoir and the core container; and a downstream pump in fluid communication with the downstream reservoir and comprising a second downstream valve for selectively permitting or ceasing fluid flow between the downstream pump and the downstream reservoir, wherein the determining comprises: installing the caprock core sample in the core container, wherein the core container is pressurized using the confining pump to a predetermined confining pressure and the caprock core sample is saturated with water; preparing the testing apparatus by closing the first upstream valve and the second downstream valve, pressurizing the upstream reservoir using the upstream pump, and closing the second upstream valve upon reaching a predetermined pressure in the upstream reservoir; conducting a flow test by opening the first upstream valve and measuring the pressure difference between a measured pressure of the upstream inlet of the caprock core sample and a measured pressure of the downstream inlet of the caprock sample; calculating a pressure differential as a function of time (ΔPₜ) based on the measured pressure difference when the pressure difference does not change with time any longer; and calculating a threshold hydraulic gradient (Jₜ), where

$${J}_{t} = \frac{Δ {P}_{t}}{L}$$

and L is a length of the caprock core sample.

In another aspect, the present disclosure provides a testing apparatus comprising: a core container for receiving a core sample comprising: an upstream inlet in fluid communication with the core container; a downstream inlet in fluid communication with the core container; and a confining pressure pump in fluid communication with a bottom portion of the core container; an upstream reservoir in fluid communication with the upstream inlet of the core container and comprising a first upstream valve for selectively permitting or ceasing fluid flow between the upstream reservoir and the core container; an upstream pump in fluid communication with the upstream reservoir and comprising a second upstream valve for selectively permitting or ceasing fluid flow between the upstream pump and the upstream reservoir; a downstream reservoir in fluid communication with the downstream inlet of the core container and comprising a first downstream valve for selectively permitting or ceasing fluid flow between the downstream reservoir and the core container; and a downstream pump in fluid communication with the downstream reservoir and comprising a second downstream valve for selectively permitting or ceasing fluid flow between the downstream pump and the downstream reservoir.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 provides a schematic drawing of gaseous (e.g., CO₂ or H₂) leakage pathways in a caprock of a saline aquifer.

FIG. 2 is a chart showing non-Darcy's flow through caprocks according to one or more aspects of the present disclosure.

FIG. 3 shows a diagram of a testing apparatus according to one or more aspects of the present disclosure.

FIG. 4 is a chart for determining the threshold hydraulic pressure using the testing apparatus according to one or more aspects of the present disclosure.

FIG. 5 is a method flowchart for determining the threshold hydraulic pressure using the testing apparatus according to one or more aspects of the present disclosure.

### DETAILED DESCRIPTION

The present disclosure relates generally to methods and systems for use in the oil and gas industry, and more particularly, to methods and systems for CO₂ and H₂ sequestration and storage.

As discussed above, there is increasing interest in systems and methods for introduction of carbon dioxide (CO₂), hydrogen (H₂), and other gases to a subterranean formation reservoir for sequestration and storage. The present disclosure provides a laboratory testing apparatus, method, and system for evaluating caprock integrity to mitigate gaseous leakage. More specifically, the present disclosure provides a system and method for evaluating caprock integrity based on measuring the threshold hydraulic gradient of caprock.

In one or more aspects, the CO₂ and H₂ may be sequestered or stored in a subterranean formation below caprock separately or in combination. For example, CO₂ may be initially injected into a reservoir followed by injection of H₂ through the same injection well. Alternately, separate injection wells may be utilized for introduction of CO₂ and H₂. It is to be noted that the CO₂, H₂, and combinations of CH₂/H₂ may be injected in one or more injection wells (i.e., CO₂ may be introduced in two or more separate wells, H₂ may be introduced in two or more separate wells, and combinations of CO₂/H₂ may be introduced in two or more separate wells). Advantageously, the methods and apparatuses of the present disclosure allow the CO₂ and H₂ sequestration and storage within subterranean formation reservoirs, such as saline aquifers and depleted oil and gas wells. Saline aquifers are generally characterized by great depths that make them often technically and economically unfeasible for exploitation for surface uses; depleted oil and gas wells have previously been drilled into a subterranean formation and can no longer be used for hydrocarbon recovery. Accordingly, advantageously, these subterranean formation reservoirs are either readily available (e.g., saline aquifers) or afford utilization of the large capital expenditures borne during hydrocarbon drilling.

Embodiments of the present disclosure are described in more detail hereinafter with reference to the accompanying Figures. In the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

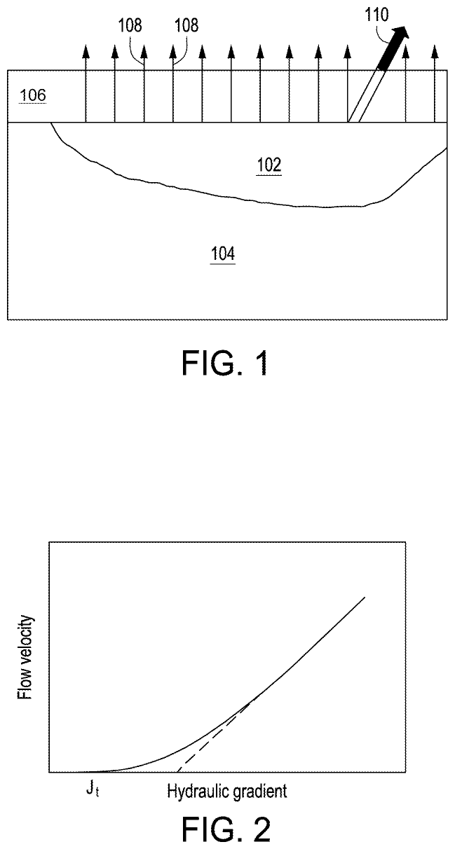

Referring to FIG. 1, illustrated is a schematic drawing of gaseous (e.g., CO₂ or H₂) leakage pathways in a caprock of a saline aquifer. As shown, a saline aquifer 104 comprises a gaseous plume 102 below caprock 106. Various gaseous leakage may potentially include area leakage 108 (the vertical arrows) or leakage through fractures and faults 110. The area leakage 108 and fracture/fault leakage 110 may provide pathways for gaseous escape through the caprock 106 due to, for example, pore pressure increase owing to the gaseous plume 102 injected into the saline aquifer 104 below the caprock 106. Area leakage 108 occurs when the stored gaseous plume 102 imbibes into the caprock 106 due to such pore pressure. The existence of fracture/fault leakage 110 occurs due to an existing fracture/fault or due to shifts in the caprock 106 after the gaseous plume 102 has been injected. Both types of leakage can lead to significant gaseous escape into the atmosphere.

More specifically, the gaseous plume 106 is non-wetting, whereas the saline aquifer 104 is wetting (water) and area leakage 108 can occur due to the pore pressure difference between the non-wetting phase and the wetting phase exceeding the break-through pressure of the caprock 106, with the break-through pressure being related to the capillary barrier between the two phases. This area leakage 108 is considered to occur by assuming that the vertical flow through the caprock 106 follows Darcy's law in which flow velocity through the caprock 106 is linearly proportional to the hydraulic gradient.

In some instances, however, flow through tight formations, such as caprock, may not follow Darcy's law due to strong water-solid interactions. Referring now to FIG. 2, illustrated is a chart showing characteristics of non-Darcy's flow through caprocks. As shown, the flow does not occur until the hydraulic gradient exceeds the threshold hydraulic gradient, labeled JI (solid line) in FIG. 2. In such instances, stored gases below the caprock may not imbibe through the caprock even after the pore pressure exceeds the breakthrough pressure. Thus, to evaluate the threshold gradient and associated non-Darcy's flow through caprock may allow for a more accurate assessment of caprock integrity for geological sequestration and storage of gases, including CO₂ and H₂ by measuring the Jₜ value.

With continued reference to FIG. 2, because of the complex and slow non-Darcy's flow character, measuring the threshold hydraulic gradient (Jₜ) of caprocks is currently rare in the oil and gas industry. Available threshold hydraulic gradient measurement techniques involve steady-state flow methods in which velocity values from caprock core samples are tested by running water therethrough with multiple test runs being necessary. Essentially, the available traditional method constructs a flow velocity as a function of hydraulic gradient to determine the threshold hydraulic gradient therefrom. Accordingly, the traditional evaluation of threshold hydraulic gradient is time-consuming and not suitable for routine use in practical applications. Differently, the methodology described herein is based on transient water flow and requires only a single test run, thus making it efficient for practical use.

Indeed, the methods and testing apparatuses of the present disclosure do not need a limited size of dead volumes because under the steady-state flow condition, the dead volumes (connected to the core sample) do not impact the flow process within the core sample. Further, methods and testing apparatuses disclosed herein allow for in-lab measurement of the permeability of a core sample as a function of pore pressure with a single test run. The results from the single in-lab test may be used to determine production characteristics of the formation from which the core sample was taken.

The methods and apparatuses of the present disclosure to evaluate and ensure caprock integrity analysis described herein, can contribute to a reduction in greenhouse gas emissions. CO₂ is a heat-trapping gas that is known to contribute to climate change, warming the climate which can lead to changing water patterns (e.g., droughts, floods), ice cap melting, heat waves, and the like. Sequestration of CO₂ accordingly can prevent or reduce it from escaping into the atmosphere.

H₂ may be considered an indirect greenhouse gas, which can react with other greenhouse gases to contribute to global warming. Accordingly, the methods and apparatuses described herein can effectively evaluate caprock for H₂ long-term storage and leakage prevention or reduction into the atmosphere. However, it can also be a vital component for decarbonization and sustainable energy systems and can capture certain greenhouse gases that are more prevalent than others, such as CO₂. The storage of H₂ in subterranean formation reservoirs can permit its later retrieval for such applications, among others.

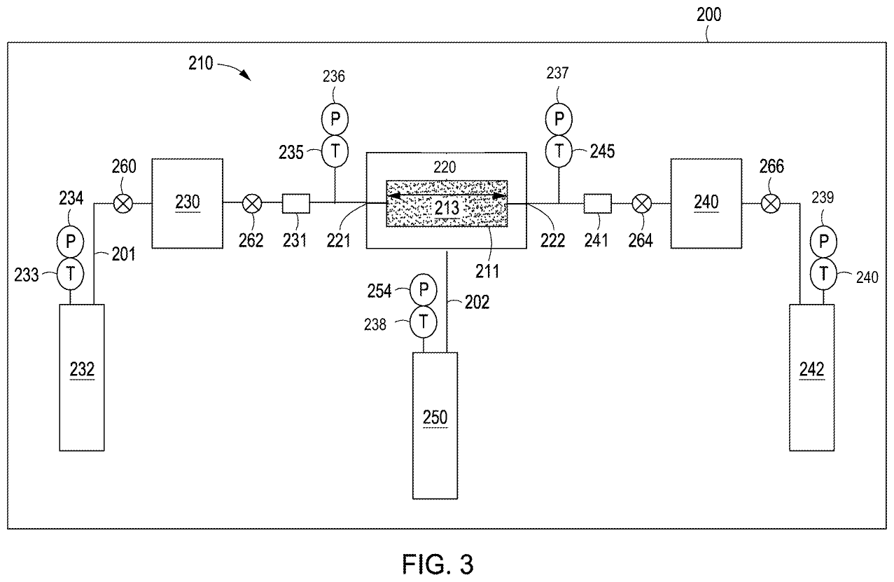

Referring now to FIG. 3, illustrated is an example testing apparatus 210 according to one or more aspects of the present disclosure, which is used to determine threshold hydraulic gradient in caprock for determining caprock integrity. Measurements are taken during a single test run as fluid is flowed through a caprock core sample 211. The testing apparatus 210 includes a core container 220 for holding the caprock core sample 211, an upstream reservoir 230, and a downstream reservoir 240, which are each connected to two different pumps to provide fluid pressures. Pump 250 is connected to core holder to provide confining pressure.

The upstream reservoir 230 may be fluidly connected to an inlet 221 of the core container 220, which is fluidly connected to an inlet side of the caprock core sample 211. Likewise, the downstream reservoir 240 may be fluidly connected to an outlet 222 of the core container 220, which is fluidly connected to the outlet side of the caprock core sample 211. The distance between the core container 220 inlet 221 and the caprock core sample 211 may vary without affecting measurements and calculations according to the embodiments of the present disclosure, as the flow path therebetween (e.g., a connecting tubing) has negligible flow resistance compared with flow in the caprock core sample 211. Thus, measurements and calculations taken in accordance with methods disclosed herein utilizing the testing apparatus 210 may interchangeably refer to confining pressure measurements at the core container 220 inlet 221 and at the inlet side of the caprock core sample 211. Similarly, because any flow path between the core container 220 outlet 222 and the outlet side of the caprock core sample 211 would have negligible flow resistance compared with flow in the caprock core sample 211, measurements and calculations taken in accordance with methods disclosed herein may interchangeably refer to pressure measurements at the core container 220 outlet 222 and at the outlet side of the caprock core sample 211.

The upstream reservoir 230 may be filled with a fluid (e.g., liquid (water) or gas). As used herein, a fluid may refer to a liquid or a gas unless stated otherwise. An upstream pump 232 may be connected to the upstream reservoir 230 to pump the fluid from the upstream reservoir 230 into the caprock core sample 211 within the core container 220. A downstream pump 242 may be connected to the downstream reservoir 240 to pump fluid (e.g., liquid (water) or gas) between the caprock core sample 211 within the core container 220 and the downstream reservoir 240, where the downstream reservoir 240 may be used to collect the fluid flowing out of the caprock core sample 211 within the core container 220 and/or to provide a back pressure to the outlet 222.

The upstream pump 232 and downstream pump 242 may be, for example, a hydraulic pump or other pump having high accuracy and high resolution, and may include precise pressure and flowrate control and measurement. The upstream pump 232 and the downstream pump 242 may together be used to control fluid flow through the caprock core sample 211 within the core container 220, including the pressure and flow rate of the fluid through the caprock core sample 211. For example, the testing apparatus 210 may include flow meters 231, 241, which may be positioned upstream the inlet 221 and downstream the outlet 222, respectively, to measure the mass flow rates at the inlet 221 and outlet 222 of the caprock core sample 211 within the core container 220. In some embodiments, the upstream pump 232 and/or the downstream pump 242 may be provided with a flow meter to measure the flow rates from the respective pumps (not shown).

A confining pump 250 may be connected to the core container 220 to apply a confining pressure around the caprock core sample 211. For example, the caprock core sample 211 may be placed in a sample cell, which may surround the caprock core sample 211 by an enclosed sleeve having an inlet side in fluid communication with the inlet 221 and an outlet side in fluid communication with the outlet 222. The enclosed caprock core sample 211 may be positioned within the core container 220, such that fluid may be flowed from the upstream reservoir 230 through the enclosed caprock core sample 211. The confining pump 250 may pump a confining fluid (e.g., a water-based fluid, an oil-based fluid, or gas) into the core container 220 around the enclosed caprock core sample 211.

Further, a plurality of pressure sensors may be used to monitor the pressure conditions at different locations in the testing apparatus 210 during the test. Pressure sensors may include high accuracy pressure transducers, including for example, piezoelectric pressure sensors, strain gauge pressure transducers, capacitance pressure transducers, potentiometric pressure transducers, and the like.

For example, an inlet pressure sensor 236 may be positioned at or proximate the inlet 221, and an outlet pressure sensor 243 may be positioned at or proximate the outlet 222.

The flow length 213 of the caprock core sample 211 is measured along the dimension of the caprock core sample 211 parallel to the direction of the flow of fluid through the caprock core sample 211 during testing. For example, when fluid is flowed through an axial length of a caprock core sample 211 during testing, the flow length 213 of the caprock core sample 211 is the axial length of the caprock core sample 211. As shown in FIG. 3, the caprock core sample 211 may be aligned axially along the axial length of the caprock core sample 211 between the inlet 221 and outlet 222, such that during testing, fluid may flow from the inlet 221, through the axial length dimension of caprock the core sample 211 (the flow length 213), and out the outlet 222.

Pump pressure sensors 234, 254, 239 may also be provided on each of the upstream pump 232, confining pump 250, and downstream pump 242, respectively, to measure the pressures of the pumps.

The testing apparatus 210 may also include a plurality of temperature sensors to monitor the temperature conditions at different locations of the testing apparatus 210 during testing. For example, an inlet temperature sensor 235 may be positioned at or proximate to the inlet 221, an outlet temperature sensor 245 may be positioned at or proximate to the outlet 222, and/or pump temperature sensors 233, 238, 240 may be provided on each of the upstream pump 232, confining pump 250, and downstream pump 242, respectively, to measure the temperature of the pumps during operation.

The testing apparatus 210 may comprise a valve 260 between the upstream pump 232 and the upstream reservoir 230 along flow line 201, a valve 262 between the upstream reservoir 230 and the flow meter 231 along flow line 201 (or optionally between the flow meter 231 and the core container 220 along flow line 201), a valve 264 between the flow meter 241 and the downstream reservoir 240 along flow line 201, and a valve 266 between the downstream reservoir 240 and the downstream pump 242. Each of valves 260, 262, 264, and 266 may be opened or closed to allow or prevent flow, respectively, through the caprock core sample 211 within the core container 220. It is to be noted that either one or both of valves 264 and 266 may be included, without departing from the scope of the present disclosure.

Optionally, the testing apparatus 210 may be placed in an oven 200 with a constant temperature such that permeability measurement tests may be conducted in an isothermal condition. In some embodiments, the oven 200 may be set to a temperature corresponding with a downhole temperature of a formation of interest, from which the core sample 211 was taken.

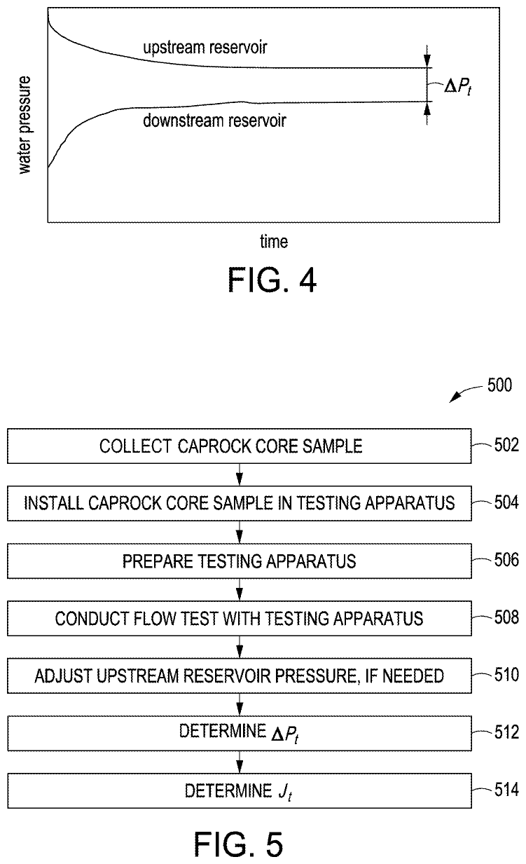

Accordingly, the testing apparatus of the present disclosure includes a caprock core container when a caprock core sample is located and subjected to confining pressure provided by confining fluid, where the upstream and downstream reservoirs are connected to the caprock core container for flow through the caprock core sample. More specifically, a fluid (e.g., gas or water) is injected from the upstream reservoir through the inlet and out of the outlet to the downstream reservoir until the caprock core sample is saturated with the fluid and reached a desired initial pore pressure. Thereafter, the fluid from the upstream reservoir is ceased from flowing to the caprock core sample (e.g., closing a valve from the upstream reservoir to the caprock core sample). One or more fluid pressure pulses are created in the upstream reservoir by injecting fluid and upon reaching the desired pressure, the upstream reservoir is again allowed to flow to the caprock core sample therefrom (e.g., opening the valve from the upstream reservoir to the caprock core sample). The fluid then flows from the upstream reservoir through the caprock core sample and to the downstream reservoir. As a result, the fluid pressure in the upstream reservoir decreases and the fluid pressure in the downstream reservoir increases until the pressure gradient reaches the threshold gradient and pressures in upstream and downstream reservoirs do not change any longer. FIG. 4 provides a chart for determining the threshold hydraulic pressure gradient using the testing apparatus of the present disclosure. As shown, the hydraulic gradient or pressure gradient decreases with time. The pressure differential reaches ΔPₜ, as shown in FIG. 4, used to calculate the threshold hydraulic gradient, flow stops, and the pressure difference no longer changes with time.

The threshold hydraulic gradient (Jₜ) may be calculated according to the following equation:

$${J}_{t} = \frac{Δ {P}_{t}}{L} ,$$

where L is the length of the caprock core sample.

Referring now to FIG. 5, showing a flowchart of an exemplary method 500 for use with the testing apparatus in one or more aspects of the present disclosure to determine threshold hydraulic gradient. FIG. 5 will be described with reference to FIG. 3.

As shown, in step 502, a caprock core sample 211 is collected. The caprock core sample 211 may be collected using a coring process, such as introducing a coring tool into a caprock to drill and collect the caprock core sample(s) 211. The caprock core sample 211 may be substantially cylindrical in shape and have a diameter in the range of about 1 inch (2.54 cm) to about 4 inches (10.16 cm), encompassing any value and subset therebetween, such as about 1 inch to about 2 inches, or about 2 inches to about 4 inches, or about 1.3 inches to about 1.7 inches, or about 2.3 inches to about 3.7 inches. In one or more aspects, the caprock core sample 211 may have an axial length, the axial length in the range of 1 inch (2.54 cm) to about 2 inches (5.08 cm), encompassing any value and subset therebetween, such as about 1 inch to about 1.5 inches, or about 1.5 inches to about 2 inches, or about 1.3 inches to about 1.7 inches. Other shapes and sizes may also be used without departing from the scope of the present disclosure, provided that they can be installed in the core container (e.g., the configuration and size of the testing apparatus). However, the caprock core sample 211 must have an axial length in a vertical direction because the imbibition of gases or other fluids through the caprock is along the vertical direction. In one or more aspects, the caprock core sample 211 may have a diameter of about 1 inch and an axial length of about 1 inch.

Step 504 involves installing the caprock core sample 211 in the testing apparatus 210 within the core container 220, the core container being pressurized. The core container 220 may withstand a radial confining pressure in the range of 500 psi to 10,000 psi, encompassing any value and subset therebetween, such as in the range of about 500 psi to about 1500 psi, or about 500 psi to about 2,500 psi, or about 2,500 psi to about 5,000 psi, or about 1,000 psi to about 2,500 psi, or about 5,000 psi to about 10,000 psi, or about 7,500 psi to about 10,000 psi. As described above, the caprock core sample 211 may be enclosed in a sleeve within the core container 220, which may include multiple sleeve layers, such that fluid flowing through the caprock core sample 211 and the provided confining fluid (within the sleeves) are separated. The sleeves may be separate or integrated with the core container 220, without departing from the scope of the present disclosure. A confining fluid may be introduced into the core container 220 and into the sleeve(s), thus not contacting the caprock core sample 211. In one or more aspects, the confining fluid may be injected into a bottom side of the core container (see FIG. 3) to fill the core container with confining fluid using a confining pump 250 through flow line 202, while incoming confining fluid may expel air through a top side of the core container (not shown).

The confining fluid and the sleeved caprock core sample 211 in the filled core container 220 may be locked-in to the core container 220 after no air is present in the core container 220. A confining pressure may then be applied to the caprock core sample 211 using the confining pump 250, such as by pumping more confining fluid to the space between the sleeved caprock core sample 211 and the core container 220 (e.g., within the sleeve). For example, after the core container is filled with confining fluid and no air is present, the core container is locked in, a confining pressure may be applied around (radially) the sleeved caprock core sample. The confining pressure may be in the range of about 400 psi to about 600 psi, encompassing any value and subset therebetween, such as in the range of about 400 psi to about 500 psi, or about 500 psi to about 600 psi, or about 450 psi to about 550 psi.

The upstream pump 232 and the downstream pump 242 at each end of the caprock core sample 211 are used to pump water into the caprock core sample 211 to saturate the caprock core sample 211. As provided above, confining pressure is imposed with the confining pump 250. The resultant pore pressure and confining pressure are set in such a way that they are consistent with field conditions of interest.

With continued reference to FIG. 5 and FIG. 3, step 506 involves preparation of the testing apparatus 210 for conducting flow testing. In this step 506, the upstream valve 262 is closed such that fluid communication between the upstream reservoir 230 and the caprock core sample 211 stops, thereby cutting off fluid communication between the upstream reservoir 230 and the caprock core sample 211. Additionally, downstream valve 266 between the caprock core sample 211 and the downstream pump 242 is closed, thereby cutting off fluid communication between the caprock core sample 211 and the downstream pump 242 (i.e., the downstream pump 242 cannot pull fluid from the caprock core sample 211 and into the downstream reservoir 240); valve 264 remains open. In this step 506, water is pulsed from the upstream pump 232 and into the upstream reservoir 230 until the water pressure in the upstream reservoir 230 reaches a desired value. Upon reaching the desired value, the upstream valve 260 between the upstream pump 232 and the upstream reservoir 230 is closed. The pressure differential between the two ends of the caprock core sample should be determined at this step.

Referring now to step 508, the flow test is conducted on the caprock core sample 211. Specifically, the upstream valve 262 is opened to allow the pressurized fluid in the upstream reservoir 230 to fluidly communicate with the caprock core sample. At this stage, all valves are closed, except those connected to the reservoirs and core sample. Upon opening the upstream valve 262, the reservoir pressure from the upstream reservoir 230 flows into the caprock core sample 211 and the pressure is monitored between each end of the caprock core sample 211 (i.e., the pressure differential) using pressure sensors 236 and 237.

Step 510 involves adjusting the upstream reservoir 230 pressure, if needed. More particularly, if the observed pressure difference (upstream pressure minus downstream pressure of the caprock core sample 211) decreases with time, this step is unnecessary, absent the necessity of the requirement to ensure whether the pressure difference decreases with time. If the pressure difference does not change (decrease) with time, then the pressure gradient along the caprock rock sample 211 is lower than the threshold hydraulic gradient. In this case, go back to Step 506 to further increase the upstream pressure.

Proceeding to Step 512, the ΔPₜ is calculated, when the pressure differential does not change with time, as described above. ΔPₜ is used to calculate the threshold hydraulic gradient under which flow stops, and the pressure difference no longer changes with time.

Finally, at Step 514, the threshold hydraulic gradient (Jₜ) is calculated based on the above equation provided herein. The threshold gradient provides an improved estimate of caprock integrity.

Accordingly, the present disclosure advantageously comprises methods and apparatuses for the determination of caprock integrity using a single, economical test based on threshold hydraulic gradient.

Embodiments disclosed herein include:

Embodiment A: A method comprising: collecting a caprock core sample; determining threshold hydraulic gradient for the caprock core sample using a testing apparatus, the testing apparatus comprising: a core container comprising: an upstream inlet in fluid communication with the core container; a downstream inlet in fluid communication with the core container; and a confining pressure pump in fluid communication with a bottom portion of the core container; an upstream reservoir in fluid communication with the upstream inlet of the core container and comprising a first upstream valve for selectively permitting or ceasing fluid flow between the upstream reservoir and the core container; an upstream pump in fluid communication with the upstream reservoir and comprising a second upstream valve for selectively permitting or ceasing fluid flow between the upstream pump and the upstream reservoir; a downstream reservoir in fluid communication with the downstream inlet of the core container and comprising a first downstream valve for selectively permitting or ceasing fluid flow between the downstream reservoir and the core container; and a downstream pump in fluid communication with the downstream reservoir and comprising a second downstream valve for selectively permitting or ceasing fluid flow between the downstream pump and the downstream reservoir, wherein the determining comprises: installing the caprock core sample in the core container, wherein the core container is pressurized using the confining pump to a predetermined confining pressure and the caprock core sample is saturated with water; preparing the testing apparatus by closing the first upstream valve and the second downstream valve, pressurizing the upstream reservoir using the upstream pump, and closing the second upstream valve upon reaching a predetermined pressure in the upstream reservoir; conducting a flow test by opening the first upstream valve and measuring the pressure difference between a measured pressure of the upstream inlet of the caprock core sample and a measured pressure of the downstream inlet of the caprock sample; calculating a pressure differential as a function of time (ΔPₜ) based on the measured pressure difference when the pressure difference does not change with time any longer; and calculating a threshold hydraulic gradient (Jₜ), where

$${J}_{t} = \frac{Δ {P}_{t}}{L}$$

and L is a length of the caprock core sample.

Embodiment B: A testing apparatus comprising: a core container for receiving a core sample comprising: an upstream inlet in fluid communication with the core container; a downstream inlet in fluid communication with the core container; and a confining pressure pump in fluid communication with a bottom portion of the core container; an upstream reservoir in fluid communication with the upstream inlet of the core container and comprising a first upstream valve for selectively permitting or ceasing fluid flow between the upstream reservoir and the core container; an upstream pump in fluid communication with the upstream reservoir and comprising a second upstream valve for selectively permitting or ceasing fluid flow between the upstream pump and the upstream reservoir; a downstream reservoir in fluid communication with the downstream inlet of the core container and comprising a first downstream valve for selectively permitting or ceasing fluid flow between the downstream reservoir and the core container; and a downstream pump in fluid communication with the downstream reservoir and comprising a second downstream valve for selectively permitting or ceasing fluid flow between the downstream pump and the downstream reservoir.

Embodiment A may have one or more of the following additional elements in any combination:

Element 1: further comprising repeating the preparing and conducting steps if the measured pressure difference between the inlet and the outlet has not changed with time.

Element 2: wherein installing the caprock core container comprises: enclosing at least one sleeve about the caprock core sample.

Element 3: wherein installing the caprock core container comprises: enclosing at least one sleeve about the caprock core sample, and wherein the core container is pressurized using the confining pump by pumping a confining fluid into core container outside of the at least one sleeve.

Element 4: wherein the upstream pump in the testing apparatus pumps water or a saline solution into the upstream reservoir and the downstream pump in the testing apparatus pumps water or a saline solution into the downstream reservoir.

Element 5: wherein the upstream pump in the testing apparatus pumps a liquid into the upstream reservoir and the downstream pump in the testing apparatus pumps a liquid into the downstream reservoir.

Element 6: wherein the caprock core sample has a diameter in the range of 1 inch to 4 inches, and an axial length in the range of 1 inch to 2 inches.

Element 7: wherein the caprock core sample has a diameter of 1 inch and an axial length of 1 inch.

Element 8: wherein the predetermined confining pressure is in the range of 500 psi to 5,000 psi.

Element 9: wherein the predetermined confining pressure is in the range of 500 psi to 2,500 psi.

Element 10: wherein the caprock core sample is collected from a saline aquifer, the saline aquifer for sequestration of CO₂ or storage of H₂.

Element 11: wherein the caprock core sample is collected from a depleted oil and gas well, the oil and gas well for sequestration of CO₂ or storage of H₂.

By way of non-limiting example, exemplary combinations applicable to A may include, but are not limited to, any one, more, or all of Elements 1-11.

Embodiment B may have one or more of the following additional elements in any combination:

Element 12: further comprising a temperature sensor provided at each of the upstream pump, downstream pump, and confining pump.

Element 13: further comprising a pressure sensor provided at each of the upstream pump, downstream pump, and confining pump.

Element 14: further comprising a temperature sensor provided proximate to each of the upstream outlet and the downstream outlet.

Element 15: further comprising a pressure sensor provided proximate to each of the upstream outlet and the downstream outlet.

Element 16: further comprising a flow meter interposing the upstream reservoir and the core container.

Element 17: further comprising a flow meter interposing the downstream reservoir and the core container.

By way of non-limiting example, exemplary combinations applicable to B may include, but are not limited to, any one, more, or all of Elements 12-17.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains,” “containing,” “includes,” “including,” “comprises.” and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized that these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and are not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

What is claimed is:

1. A method comprising:
collecting a caprock core sample;
determining threshold hydraulic gradient for the caprock core sample using a testing apparatus, the testing apparatus comprising:
a core container comprising:
an upstream inlet in fluid communication with the core container;
a downstream inlet in fluid communication with the core container; and
a confining pressure pump in fluid communication with a bottom portion of the core container;
an upstream reservoir in fluid communication with the upstream inlet of the core container and comprising a first upstream valve for selectively permitting or ceasing fluid flow between the upstream reservoir and the core container;
an upstream pump in fluid communication with the upstream reservoir and comprising a second upstream valve for selectively permitting or ceasing fluid flow between the upstream pump and the upstream reservoir;
a downstream reservoir in fluid communication with the downstream inlet of the core container and comprising a first downstream valve for selectively permitting or ceasing fluid flow between the downstream reservoir and the core container; and
a downstream pump in fluid communication with the downstream reservoir and comprising a second downstream valve for selectively permitting or ceasing fluid flow between the downstream pump and the downstream reservoir,
wherein the determining comprises:
installing the caprock core sample in the core container, wherein the core container is pressurized using the confining pump to a predetermined confining pressure and the caprock core sample is saturated with water;
preparing the testing apparatus by closing the first upstream valve and the second downstream valve, pressurizing the upstream reservoir using the upstream pump, and closing the second upstream valve upon reaching a predetermined pressure in the upstream reservoir;
conducting a flow test by opening the first upstream valve and measuring the pressure difference between a measured pressure of the upstream inlet of the caprock core sample and a measured pressure of the downstream outlet of the caprock sample;
calculating a pressure differential as a function of time (ΔPₜ) based on the measured pressure difference when the pressure difference does not change with time any longer; and
calculating a threshold hydraulic gradient (Jₜ), where
$${J}_{t} = \frac{Δ {P}_{t}}{L}$$
and L is a length of the caprock core sample.

2. The method of claim 1, further comprising repeating the preparing and conducting steps if the measured pressure difference between the inlet and the outlet has not changed with time.

3. The method of claim 1, wherein installing the caprock core container comprises:
enclosing at least one sleeve about the caprock core sample.

4. The method of claim 3, wherein the core container is pressurized using the confining pump by pumping a confining fluid into core container outside of the at least one sleeve.

5. The method of claim 1, wherein the upstream pump in the testing apparatus pumps water or a saline solution into the upstream reservoir and the downstream pump in the testing apparatus pumps water or a saline solution into the downstream reservoir.

6. The method of claim 1, wherein the upstream pump in the testing apparatus pumps a liquid into the upstream reservoir and the downstream pump in the testing apparatus pumps a liquid into the downstream reservoir.

7. The method of claim 1, wherein the caprock core sample has a diameter in the range of 1 inch to 4 inches, and an axial length in the range of 1 inch to 2 inches.

8. The method of claim 1, wherein the caprock core sample has a diameter of 1 inch and an axial length of 1 inch.

9. The method of claim 1, wherein the predetermined confining pressure is in the range of 500 psi to 5,000 psi.

10. The method of claim 1, wherein the predetermined confining pressure is in the range of 500 psi to 2,500 psi.

11. The method of claim 1, wherein the caprock core sample is collected from a saline aquifer, the saline aquifer for sequestration of CO₂ or storage of H₂.

12. The method of claim 1, wherein the caprock core sample is collected from a depleted oil and gas well, the oil and gas well for sequestration of CO₂ or storage of H₂.

13. A testing apparatus comprising:
a core container for receiving a core sample comprising:
an upstream inlet in fluid communication with the core container;
a downstream inlet in fluid communication with the core container; and
a confining pressure pump in fluid communication with a bottom portion of the core container;
an upstream reservoir in fluid communication with the upstream inlet of the core container and comprising a first upstream valve for selectively permitting or ceasing fluid flow between the upstream reservoir and the core container;
an upstream pump in fluid communication with the upstream reservoir and comprising a second upstream valve for selectively permitting or ceasing fluid flow between the upstream pump and the upstream reservoir;
a downstream reservoir in fluid communication with the downstream inlet of the core container and comprising a first downstream valve for selectively permitting or ceasing fluid flow between the downstream reservoir and the core container; and
a downstream pump in fluid communication with the downstream reservoir and comprising a second downstream valve for selectively permitting or ceasing fluid flow between the downstream pump and the downstream reservoir.

14. The testing apparatus of claim 13, further comprising a temperature sensor provided at each of the upstream pump, downstream pump, and confining pump.

15. The testing apparatus of claim 13, further comprising a pressure sensor provided at each of the upstream pump, downstream pump, and confining pump.

16. The testing apparatus of claim 13, further comprising a temperature sensor provided proximate to each of the upstream outlet and the downstream outlet.

17. The testing apparatus of claim 13, further comprising a pressure sensor provided proximate to each of the upstream outlet and the downstream outlet.

18. The testing apparatus of claim 13, further comprising a flow meter interposing the upstream reservoir and the core container.

19. The testing apparatus of claim 13, further comprising a flow meter interposing the downstream reservoir and the core container.
